(19) United States
(12) Patent Application Publication
(10) Pub. No.: US 2024/0274030 A1
(43) Pub. Date: Aug. 15, 2024

(54) Smash Intervention Technologies

(71) Applicant: Christina Toliver, St Petersburg, FL (US)

(72) Inventor: Christina Toliver, St Petersburg, FL (US)

(21) Appl. No.: 18/166,683

(22) Filed: Feb. 9, 2023

(51) Int. Cl. G09B 19/00; G16H 10/20; G16H 20/70

(52) U.S. Cl. CPC G09B 19/00; G16H 10/20; G16H 20/70

(57) ABSTRACT

Smash Therapy is a Method of Mental Health Therapy that combines smashing and psychotherapy delivered via traditional smashing as offered by rage rooms and includes the guided assistance of a psychotherapist to assist in the release of pent up frustration. The second method of delivery of Smash Therapy is through a virtual reality Smash Therapy metaverse that is being developed based on ongoing research and development and includes the Smash Therapy methodology accessible via any Virtual Reality mechanism, and the third method of delivery is through our Mobile Smash Clinics, which delivers Smash Therapy directly to the client location. Smash Therapy includes frequency, duration, and stimulation guidelines that formulate effective delivery of this method of mental health therapy collected through surveys conducted with previous clients incorporated into the design and testing is ongoing.

### BACKGROUND OF THE INVENTION

SmashBox20 is a woman-owned and operated small business in Pinellas County, Florida that was started in January 2021 to facilitate the smashing activity; a service offered by rage rooms that allows participants to break things in an avowedly safe environment to relieve stress and frustration. Through our experience serving over twenty-seven hundred customers over the last two years and collaboration with the University of South Florida, the NSF IUCRC C2SHIP program, the College of Nursing and BIOS Institute, the Pinnacle and C2SHIP UA Director of Translation Health Science, IRB coordinator, and Begin Again Counseling Services, a mental health clinic with twelve staff psychotherapists and licensed behavioral health counselors, we claim to have developed “Smash Intervention Technologies”. Smash Intervention Technologies, a novel mental health therapy and intervention that involves specialized delivery methods of smashing that includes Psychotherapist Guided Smashes, Virtual Reality Metaverse Smashing Environments, and a Mobile Smash Clinic. In an ongoing effort to enhance these delivery methods, Smash Intervention Technologies also involves a proprietary wearable sensor device to monitor participants' response and tracks health information to the smashing activities. The sensor technology adds a combination of gaps, barriers, and wicking textile materials to tune and modulate the response to Smash Intervention Technologies, allowing therapists to monitor heartrate, sweat, and movement to enhance delivery and outcomes of Smash Intervention Technologies. The technology uses real-time sensor data with sweats, heartbeats, and movement record through graph neural network with LSTM structures and variational inference features. The LSTM will also help train AI to understand the sequence of events, such as conversion of raging behaviors and whether symptoms improve or worsen post-smashing, and the physiological status in time series. The variational inference features help to personalize the AI/ML models to each user.

### DETAILED DESCRIPTION OF THE INVENTION

### Smash Intervention Technologies

Psychotherapy provides an invaluable resource and outlet for individuals experiencing mental health issues; however, it does not provide the ability to physically release associated negative toxins our bodies retain when we experience symptoms from mental illness like smashing does. Comparable to intense exercise as it relates to modulation of neurotransmitters such as dopamine (DA), noradrenaline (NE), and serotonin (5-HT), Smashing is a euphoric exercise that allows an individual to project negative emotions, thoughts, and feelings onto inanimate objects that by smashing items, simulate the destruction of those inhibitors, providing the individual the ability to release and let go of what is adversely affecting and impeding on their mental health from improving.

The concept of rage rooms originated in Japan around 2008 and has become very popular throughout the world since then. Rage rooms facilitate the smashing activity in an avowedly safe environment, allowing patrons to break items such as glass, furniture, and electronics, using bats, golf clubs, and sledgehammers to relieve stress and frustration (Lyford, 2021). Several studies have found a positive relationship between smashing activities and improvements of depression among individuals with osteoarthritis (Wi, Kang, & Jang, 2013), physical and mental health for cancer rehabilitation patients (Person, Clifford, Wallergard, & Sanden, 2021), and reduced work stress/psychological complaints among working population (Brzozowski, 2019).

Building upon existing concepts of a rage room, psychotherapy, and technology, SmashBox20 has developed “Smash Intervention Technologies”, a new intervention model which combines the smashing activity and psychotherapy to provide an alternative treatment option for mental health issues such as depression, anxiety, and relational concerns with family. This novel design includes smashing activities with optional onsite psychotherapy to provide a cost-effective intervention for individuals who may not benefit from traditional talk therapy. Smash Intervention Technologies offers a guided smash exercise along with an in-session psychotherapist that instructs clients on how to release pent up emotions throughout their smash session. In addition, our company is developing wearable sensor devices to monitor participants' response and track health information to the smashing activities. The technology adds a combination of gaps, barriers, and wicking textile materials to tune and modulate the response, allowing therapists to monitor heartrate, sweat, and movement. Smash Intervention Technologies also offers simulated virtual reality metaverse smash therapy environments offering maximum safety and accessibility, especially for individuals who suffer from mobility issues or certain disabilities that can make the physical smashing activity inaccessible. Mobile On-Site Smash Clinics is a third delivery method of Smash Intervention Technologies, which is SmashBox20's Mobile Smash Unit that provides all-inclusive, on-site traditional smashing at mental health clinics, corporate offices, private homes, and any permissible location, eliminating the need to come to brick-and-mortar locations to participate. In summary, the major components of Smash Intervention Technologies include:
A Guided Smash Intervention Technologies protocol and manual
A wearable controller that integrates the sensor pad and emits via Bluetooth (BLE)
A portable desktop base-station to acquire BLE and forward data to a cloud-server
A virtual metaverse smash program
A mobile smash clinic

Guided Smash Session: R&D efforts are underway to further develop this protocol. A pilot study is on-going to test various types of combinations of guided smash sessions with therapy.

Portal for therapists: This is a critical area requiring collaboration with a smash room and counseling facility. As with the data visualization, much can be accomplished in this phase (prior to an IRB). The web portal dashboard can be reconfigured to therapists' usages through a new belief inference tree running in the background.

Artificial Intelligence/Machine learning: The team will correlate real-time sensor data with sweats, heartbeats, and movement record through graph neural network with LSTM structures and variational inference features. The LSTM will help train AI to understand the sequence of events, such as conversion of raging behaviors and whether symptoms improve or worsen post-smashing, and the physiological status in time series. The variational inference features help to personalize the AI/ML models to each user.

Our focus on the advanced development of intervention protocol and monitoring sensor system will assure that we work both standalone, while being able to contribute a solid component to a more expansive mental health system. In summary, the above areas require significant time and efforts as we move on to create the basis for a commercial product.

### REFERENCES

Anxiety and Depression Association of America. (2021, April 21). Facts & statistics. ADAA. https://adaa.org/understanding-anxiety/facts-statistics
Lyford, C. (2021, November). Rage Rooms. Psychotherapy Networker, 45, 11-13. https://www.proquest.com/magazines/rage-rooms/docview/2598124619/se-2
Zelis C. & Borden, E. (2021, July 28). The growing importance of EAPS as Americans head back to the Office. BenefitsPRO. Retrieved Feb. 9, 2023, from https://www.benefitspro.com/2021/07/28/the-growing-importance-of-eaps-as-americans-head-back-to-the-office/
Brzozowski, C. (2019, March-April). Rage Rooms Offer a Chance to Vent . . . and Smash Stuff: Although their methods have detractors, two HR practitioners are taking a swing at employee catharsis. Workforce, 98(2), 10. https://link.gale.com/apps/doc/A580474212/AONE?u=tamp44898&sid=bookmark-AONE&xid=f949e276
Mental Health Professionals: US Statistics 2017. (2019, April 9). Psych Central. https://psychcentral.com/blog/mental-health-professionals-us-statistics-2017#4
Persson, J., Clifford, D., Wallergård, M., & Sanden, U. (2021). A Virtual Smash Room for Venting Frustration or Just Having Fun: Participatory Design of Virtual Environments in Digitally Reinforced Cancer Rehabilitation. JMIR rehabilitation and assistive technologies, 8(4), e29763. https://doi.org/10.2196/29763
Wi, S. Y., Kang, J., & Jang, J. (2013). Clinical Feasibility of Exercise Game for Depression Treatment in Older Women with Osteoarthritis: a Pilot Study. Journal of Physical Therapy Science, 25, 165-167.

1. I, Christina Toliver formed SmashBox20, LLC in January 2021 and claim to have began developing Smash Therapy. I submitted the original idea of Smash Therapy to the National Institute of Mental Health on Dec. 30, 2021, inquiring about a Division of Services and Intervention Research grant to assist with Research and Development of this product and submitted the original Whitepaper that I will supply as a separate attachment. After being provided specific directives from the NIMH, I collaborated with the University of South Florida and refined Smash Therapy, a Method of Mental Health Therapy that combines smashing and psychotherapy and delivered via traditional smashing as offered by rage rooms that includes the guided assistance of a psychotherapist. The second method of delivery of Smash Therapy is through a virtual reality Smash Therapy metaverse that is being developed based on R&D, and the third method of delivery is through our Mobile Smash Clinics, which delivers Smash Therapy directly to the client. Smash Therapy also includes frequency, duration, and stimulation findings to formulate effective delivery from surveys conducted with previous clients incorporated into the design and testing is ongoing.
